(19) United States
(12) Patent Application Publication
(10) Pub. No.: US 2018/0263975 A1
(43) Pub. Date: Sep. 20, 2018

(54) Methods Of Treating Motor And Movement Disorders And Side Effects Thereof Associated with Parkinson's Disease Treatments

(71) Applicant: AMARANTUS BIOSCIENCE HOLDINGS, INC., San Francisco, CA (US)

(72) Inventors: David A. Lowe, VEVEY (CH); Charlotte Keywood, Esher Surrey (GB)

(21) Appl. No.: 15/542,286

(22) PCT Filed: Jan. 13, 2016
(86) PCT No.: PCT/US2016/013206

§ 371 Date: Jul. 7, 2017

Related U.S. Application Data
(60) Provisional application No. 62/103,036, filed on Jan. 13, 2015.

(51) Int. Cl. A61K 31/496; A61K 31/198; A61K 9/00; A61K 9/24

(52) U.S. Cl. CPC A61K 31/496; A61K 9/209; A61K 9/0053; A61K 31/198

(57) ABSTRACT

This invention provides methods of treating motor disorder side effects associated with the administration of levodopa to a subject having Parkinson's disease, by administering eltoprazine or a pharmaceutically acceptable acid addition salt thereof in doses and dosing schedules which result in beneficial antidyskinetic effects for the subject. In particular, the invention provides methods for reducing dyskinesia associated with Parkinson's disease treatments, and effective doses and dosing regimens of eltoprazine or a pharmaceutically acceptable acid addition salt thereof.

### FIELD OF THE INVENTION

This invention relates generally to the administration of drugs having agonist activity at 5-HT₁ₐ and/or 5-HT_{1b} receptors to patients in need thereof in order to prevent, attenuate and/or treat motor disorders, movement disorders and side effects thereof associated with drugs that increase the activity of the dopamine receptor, including dopamine agonists and partial agonists, whether acting directly or indirectly, as well as dopamine precursors, such as levodopa (L-DOPA). In particular, the invention relates to methods of preventing and treating L-DOPA-induced dyskinesia (LID), and to preventing and treating Parkinson's disease, by administering eltoprazine, particularly in certain doses and dosing or administration regimens, either alone or in combination with other compounds.

### BACKGROUND OF THE INVENTION

Parkinson's disease is a chronic, progressive, hypokinetic neurodegenerative disorder characterized by impaired voluntary movement (See, Dale and Federman (eds.), WebMD Scientific American Medicine, NY: WebMD Corporation, Chapter 11, Section 15, pp. 1-21,2001; Lang and Lozano, N Engl J Med, 339: 1044, 1998; and Lang and Lozano, N Engl J Med, 339: 1130, 1998). Parkinson's disease occurs at least as a result of the death of dopamine-producing neurons in the substantia nigra of the midbrain. Dopamine is a neurotransmitter, or chemical messenger, that transports signals to the parts of the brain that control movement initiation and coordination. The loss of dopamine in the brain is associated with multiple primary symptoms including, for example, tremor of the hands, arms, legs, jaw, and face; rigidity or stiffness of the limbs and trunk; bradykinesia or slowness of movement; dyskinesia; and postural instability or impaired balance and coordination. The disease is also associated with dementia, sleep disturbances and cognitive confusion.

Parkinson's disease afflicts more than one million persons in the United States alone (Lang and Lozano, supra, 1998), with approximately 50,000 new cases diagnosed each year. It is generally a disease of late middle age, with typical onset occurring at about age 60. About five percent of patients, however, have early-onset disease and are younger than 40 when symptoms begin.

Most reported treatment strategies for PD focus on symptom control through one or more of medication, surgery, and physical therapy. Administration of the dopamine precursor, L-DOPA (L-3,4-dihydroxyphenylalanine; levodopa) is reported as the most effective and most commonly used treatment for PD, as it reverses the motor deficits associated with PD more so than dopamine agonists (Goodman & Gillman's The Pharmacological Basis of Therapeutics, 11^{th} edition, L. Brunton editor, The McGraw Hill Company, 2006). Unfortunately, L-DOPA can cause debilitating side effects (LeWitt and Nyholm Neurology, 62:S9-S16, 2004), including severe nausea, vomiting, and psychosis. Moreover, with prolonged use, patients frequently experience other side effects such as dyskinesia, or abnormal, excessive movements in many patients, which can interfere with the management of PD (see, e.g., Baldessarini R J., Am. J. Psychiatry, 137: 1163-72 (1980); Samii et al., Lancet, 363(9423): 1783-93 (2004)).

Serotonin neurons are reported to play a role in the development of L-DOPA-induced dyskinesia (LID), and specifically, 5-HT₁ₐ and 5-HT_{1b} receptors are thought to be involved (see, e.g., Carta et al., Brain, 130(7): 1819-33 (2007); and U.S. published application 2007/0249621). In rats, activation of 5-HT₁ₐ and 5-HT_{1b} receptors, either separately, or in combination, was reported to reduce LID (Carta et al., 2007). When administered together at certain low doses, 5-HT₁ₐ and 5-HT_{1b} receptor agonists were stated to block LID in rats (Carta et al., 2007). WO2010/063486 to Merz et al., WO 2009/156380 to Bjorklund et al.; U.S. patent publication 2007/0249621 to Wolf et. al.; U.S. patent publication 2010/0179171 to Wolf et. al.; and European Patent Application No. 2193794 to Valastro et al. relate to the use of eltoprazine to treat LID in rats.

The binding profile of eltoprazine as reported by Sijbesma (Sijbesma, H. et al., European Journal of Pharmacology, 187(2): 209-223, (1990)) shows the compound to be a selective 5-HT₁ ligand (selective with respect to all receptors other than 5-HT₁) and similar to serotonin in many respects except for a lower affinity for the 5-HT_{1d} receptor. The literature reports that eltoprazine acts as a mixed 5-HT₁ₐ/5-HT_{1b} receptor agonist with roughly equipotent affinity for each of these receptors (Schipper et al., Drug Metabol Drug Interact, 8(1-2):85-114, (1990)). Eltoprazine has no relevant affinity for dopamine receptors. Among the 5-HT receptors, the 5-HT_{1b} receptor is located as an autoreceptor on axon terminals and is responsible for inhibiting neurotransmitter release, whereas it is also located postsynaptically as a heteroreceptor on axons and terminals of non-serotonergic neurons inhibiting their activity (Clark and Neumaier, Psychopharmacol Bul, 35(4): 170-85, (2001)).

There exists a need to develop noninvasive treatment methods which can effectively prevent, attenuate, control, and treat the motor disorders, movement disorders, and side effects of the movement and motor disorders associated with drug treatments for PD. There is a need to prevent, attenuate, and treat L-DOPA-induced dyskinesia (LID), as well as to prevent, attenuate, and treat Parkinson's disease (PD). In particular, there is a need to develop strategies that target serotonin receptors, including 5-HT₁ₐ and 5-HT_{1b} receptors, either in combination or separately, for the prevention, attenuation and treatment of movement disorders, such as L-DOPA-induced dyskinesia, associated with Parkinson's disease treatments. There is also a need to develop treatment strategies for controlling the symptoms of Parkinson's disease and other movement disorders and effective pharmaceutical compounds and dosages for treating these symptoms. In addition, there is a need for specific therapy regimens that maximize the efficacy, and reduce the side effects of existing medicines, for Parkinson's disease.

### SUMMARY OF THE INVENTION

This invention provides methods for preventing, attenuating, and/or treating in patients (preferably humans) with PD, movement disorders, motor disorders, or movement or motor disorder side effects associated with dopamine-related drugs, in addition to preventing, attenuating, and/or treating PD. The methods involve treating patients with eltoprazine in certain doses and dose regimens found to be effective in reducing LID in human patients, specifically when eltoprazine is administered to Parkinson's patients at particular doses and dosing intervals. In an particular embodiment, LID severity is reduced in LID patients to whom eltoprazine is dosed two or three times daily at 4 hour intervals at doses of 2.5 mg, 5 mg, 7.5 mg and 10 mg. In a particular embodiment, eltoprazine is administered to a patient to reduce LID one, two, or three times daily at a dose of 5 mg or 7.5 mg. In an embodiment, eltoprazine is orally administered. In an embodiment, eltoprazine is administered in a controlled release, sustained release, extended release, delayed release, or pulsatile release dosage form, such as, without limitation, an oral solid dosage form. In an embodiment, the oral dosage form is a multi-layered solid dosage form, such as a bi-layered or a tri-layered tablet. The invention provides effective and acutely administered doses of eltoprazine which do not affect the efficacy of levodopa treatment, do not affect the therapeutic response to levodopa and are not associated with any serious adverse events in patients undergoing treatment.

The dopamine-related drugs encompassed by the invention include drugs that increase the activity of the dopamine receptor, including dopamine agonists and partial agonists, whether acting directly or indirectly, as well as dopamine precursors, such as L-DOPA. In preferred embodiments, the invention encompasses preventing, attenuating, and/or treating motor disorder side effects, including but not limited to dyskinesia, that are associated with L-DOPA therapy in Parkinson's patients (L-DOPA-induced dyskinesia, LID). The methods of the invention comprise administering to a patient in need thereof a therapeutic dose of a compound having agonist activity at both the 5-HT₁ₐ and 5-HT_{1b} receptors (eltoprazine or batoprazine as non-limiting examples), or two separate compounds having agonist activity, one targeting the 5-HT₁ₐ receptor, and another compound targeting the 5-HT_{1b} receptor. The methods of the invention also encompass preventing, attenuating, and/or treating PD in a patient in need thereof. In particular embodiments, the 5-HT₁ₐ and 5-HT_{1b} receptor agonist is eltoprazine or eltoprazine hydrochloride.

The invention encompasses many possible administration strategies including, but not limited to, administration of the 5-HT_{1a/1b} receptor agonist or partial agonist before or after initiation of L-DOPA or other dopamine-related drug administration, and administration of the 5-HT_{1a/1b} receptor agonist or partial agonist before or after development of motor disorder side effects. Dosage strategies include therapeutic and sub-therapeutic dosages of L-DOPA or other dopamine-related drug. In certain embodiments, this invention encompasses a reduction in the dosage of L-DOPA or other dopamine-related drug after administration of the 5-HT_{1a/1b} receptor agonist or partial agonist. In instances where eltoprazine is used as the 5-HT_{1a/1b} receptor agonist, non-limiting examples of daily dosages include from 3 mg to 30 mg; or 2.5 mg, 5.0 mg, 7.5 mg, and 10 mg as embodiments within the scope of the invention. Particularly preferred dosages of eltoprazine, in humans, are 5 mg/day and 7.5 mg/day. Administration schedules may also be altered to achieve a therapeutically effective concentration of compound to treat the disorder or symptoms described herein. In some embodiments, for example, the compound may be administered once per day, twice per day, thrice per day, 4 times per day, 5 times per day, 7 times per day or 10 times per day.

The invention encompasses methods of treating levodopa (L-DOPA) induced dyskinesia (LID) in a subject, preferably a human patient, in need thereof. In an embodiment, the subject has Parkinson's disease (PD). In embodiments, the methods involve administering eltoprazine to a patient in need at a dose and dose regimen to effectively reduce or abrogate LID in the subject. In a particular embodiment, the eltoprazine dose is 5 mg or 7.5 mg. In other particular embodiments, eltoprazine is administered to the subject once, twice, or three times daily, in particular, two or three times daily. In an embodiment, eltoprazine is administered to the subject at hourly intervals of 4, 6, 8, and 12 hours, in particular, at 4 hour intervals.

In particular embodiments, the methods involve administering eltoprazine to a patient in need at a dose of 5 mg or 7.5 mg to effectively reduce dyskinesia, namely, LID. In embodiments, a daytime plasma concentration following eltoprazine administration is in the range of 9 ng/ml to 30 ng/ml; or in the range of 10 ng/ml to 30 ng/ml; or in the range of 9 ng/ml to 17 ng/ml. In particular embodiments, at a dose of 5 mg, the plasma concentration of drug (eltoprazine) is approximately 10 ng/ml, while at a dose of 7.5 mg, the plasma concentration of drug (eltoprazine) is approximately 17 ng/ml. The invention encompasses a variety of daily dosing combinations to achieve a desired and effective target daytime plasma concentration range. Oral dosing and administration is encompassed by the methods.

The methods of the invention embrace a number of dosing combinations and regimens involving eltoprazine to reduce LID and/or to optimize the daytime plasma concentration in the individual (human patient) undergoing treatment. In an embodiment, an individual in need is dosed twice daily at four or six hourly intervals with an eltoprazine dose of 2.5 mg, 5 mg, or 7.5 mg. In a particular embodiment, the dose is 5 mg. In another particular embodiment, the dose is 7.5 mg. In an aspect, dosing at four or six hour intervals may optimize the daytime plasma concentration in the individual from 10 AM to 8 PM, for example. Oral dosing and administration of eltoprazine are embraced by this dosing combination and regimen.

In an embodiment related to dosing and dosing regimens of eltoprazine, an individual (human patient) in need is dosed twice daily at four hour intervals with an eltoprazine dose of 2.5 mg, 5 mg, 7.5 mg, or 10 mg. In a particular embodiment, the dose is 5 mg. In another particular embodiment, the dose is 7.5 mg. In an aspect, eltoprazine doses of 5 mg and 7.5 mg may optimize the daytime plasma concentration, for example, from 10 AM to 8 PM. Oral dosing and administration of eltoprazine are embraced by this dosing combination and regimen.

In another embodiment related to dosing and dosing regimens of eltoprazine, an individual (human patient) in need is dosed three times daily at four hour intervals with an eltoprazine dose of 2.5 mg, 5 mg, or 7.5 mg. In a particular embodiment, the dose is 5 mg. In another particular embodiment, the dose is 7.5 mg. In an aspect, three doses of eltoprazine, such as a 5 mg dose and a 7.5 mg dose, may allow maximal plasma concentrations to be achieved, for example, in the late afternoon evening period, extending into the night time. Oral dosing and administration of eltoprazine are embraced by this dosing combination and regimen.

In another embodiment, an individual in need (human patient) may be given a morning loading dose of eltoprazine, followed by another dose at a predetermined interval, such as at midday, with a total daily dose of eltoprazine of 10 mg or 15 mg. Such a regimen involving a loading dose followed by one or more smaller, ‘top up’ doses, may optimize daytime exposure while minimizing the overall dose administered to the individual. In an embodiment, an individual in need is dosed with eltoprazine three times daily at four hour intervals with a dose of 2.5 mg, 5 mg, or 7.5 mg. In a particular embodiment, the dose is 5 mg thrice daily every four hours. In another particular embodiment, the dose is 7.5 mg thrice daily every four hours. Oral dosing and administration of eltoprazine are embraced by this dosing combination and regimen.

In embodiments related to oral dosing of eltoprazine and the dosing regimens herein described, the oral dosage form is not intended to be limiting and thus may include liquid oral dosing forms and solid oral dosage forms, such as pills, capsules, or tablets. In an embodiment, the dosage form is a controlled release, sustained release, extended release, delayed release, or pulsatile release dosage form. In an embodiment, the controlled release, sustained release, extended release, delayed release, or pulsatile release dosage form is an oral solid dosage form. Multilayered tablets, such as bi-layered and tri-layered tablets as controlled, sustained, delayed, extended, or pulsatile release solid dosage forms are encompassed by the invention.

Kits comprising one or more of the following are also encompassed by the invention described herein: a compound having agonist activity at both the 5-HT₁ₐ and 5-HT_{1b} receptors, or two separate compounds having agonist activity, one targeting the 5-HT₁ₐ receptor, and another compound targeting the 5-HT_{1b} receptor, additional compounds, L-DOPA or other or other dopamine-related drug, and instructions for administration. Non-limiting examples of such kits include a kit comprising eltoprazine, and a kit comprising eltoprazine, L-DOPA, a DDCI inhibitor and/or COMT inhibitor, and optionally any other compound described herein, plus instructions for administration.

### BRIEF DESCRIPTION OF THE FIGURES

FIG. 1 shows the efficacy of eltoprazine in treating levodopa-induced dyskinesia patients red using Clinical Dyskinesia Rating Scales (CDRS).

FIG. 2 shows that there is no significant change in the Unified Parkinson's Disease Rating Scales-III (UPDRS-III) of parkinsonian symptoms after eltoprazine administration, indicating that eltoprazine does not adversely interfere with the levodopa efficacy in Parkinson's patients.

FIG. 3 shows the results of twice daily dosing with 7.5 mg of eltoprazine at 4, 6, 8 and 12 hourly intervals. As observed, dosing at 4 or 6 hourly intervals optimizes daytime plasma concentration from 10 AM to 8 PM.

FIG. 4 shows the results of twice daily dosing with 2.5, 5, 7.5 and 10 mg of eltoprazine at 4 hour intervals. As observed, doses of 5 mg and 7.5 mg b.d. (twice per day), given at 4 hour intervals, optimizes daytime plasma concentration from 10 AM to 8 PM.

FIG. 5 shows the results of three times daily dosing with 2.5 mg, 5 mg, and 7.5 mg of eltoprazine at 4 hour intervals. As observed, three doses, given at 4 hour intervals, achieve maximal plasma concentrations in the late afternoon/evening period into the night time.

FIG. 6 shows the results of a morning loading dose of eltoprazine, followed by midday ‘top up’ for a total daily dose of 10 mg, or 15 mg. A loading dose followed by smaller top up doses may optimize an individual's daytime exposure to drug while minimizing the dose.

### DETAILED DESCRIPTION

This invention encompasses methods of preventing, attenuating, and/or treating motor disorder side effects in a patient with PD, including but not limited to, dyskinesia or other motor disorder, movement disorder, or motor or movement disorder side effects, that are associated with dopamine-related drugs, in addition to preventing, attenuating, and/or treating PD. In an embodiment, the methods involve therapeutic doses of a compound having agonist activity at both the 5-HT₁ₐ and 5-HT_{1b} receptors, such as eltoprazine or a pharmaceutically acceptable salt thereof, and dosing regimens and combinations which optimally provide effective and lasting plasma concentrations of drug in a patient undergoing treatment.

In an embodiment, the method comprises administering to a patient in need thereof a therapeutic dose of a compound having agonist activity at both the 5-HT₁ₐ and 5-HT_{1b} receptors, or two separate compounds having agonist activity, one targeting the 5-HT₁ₐ receptor, and another compound targeting the 5-HT_{1b} receptor. Optionally in combination with targeting the 5-HT₁ₐ and 5-HT_{1b} receptors, the invention encompasses administering to the patient in need thereof a compound that targets the same, a similar, or a different drug pathway, one that is useful in treating L-DOPA-induced dyskinesia (LID) or other movement disorders or motor disorder side effects associated with dopamine-related drugs, one that is useful in treating PD, or one that is useful for treating both LID or other movement or motor disorders, or movement or motor disorder side effects associated with dopamine-related drugs and PD. This additional compound may also be useful such that the effective dose of L-DOPA or other dopamine-related drug that is necessary to treat PD is reduced. In some embodiments, the invention contemplates administering to the patient in need thereof L-DOPA, in addition to a compound that targets the same, a similar, or a different drug pathway, one that is useful in treating L-DOPA-induced dyskinesia (LID), one that is useful in treating PD, or one that is useful for treating both LID or other movement disorders or motor disorder side effects associated with dopamine-related drugs and PD.

The dopamine-related drugs encompassed by the invention include drugs that increase the activity of the dopamine receptor, including dopamine agonists and partial agonists, whether acting directly or indirectly, as well as dopamine precursors, such as L-DOPA. In a preferred embodiment, the dopamine-related drug treatment is L-DOPA therapy. In other embodiments, the dopamine-related drug may be a dopamine receptor agonist, including, but not limited to bromocriptine, pergolide, cabergoline, apomorphine, and lisuride, or a non-ergoline dopamine agonist, including, but not limited to ropinirole or pramipexole.

In yet other embodiments, the methods of the invention encompass preventing, attenuating, and/or treating any of the movement disorders, motor disorders, movement disorder side effects, or motor disorder side effects described herein, associated with dopamine-related drug treatments, or a combination of dopamine-related drug treatments, such as a combination of dopamine precursors, a combination of dopamine agonists or partial agonists, and a combination of one or more dopamine precursors and one or more dopamine agonists or partial agonists.

Dopamine precursors such as L-DOPA are often administered with a DOPA decarboxylase inhibitor (DDCI) (also known as aromatic L-amino acid decarboxylase inhibitors (AAADI)). Non-limiting examples of such compounds contemplated by the invention include benserazide (Madopar, Prolopa, Modopar, Madopark, Neodopasol, and EC-Doparyl); carbidopa (Lodosyn, Sinemet, Parcopa, and Atamet); and Methyldopa (Aldomet, Aldoril, Dopamet, and Dopegyt). In addition to DDCIs, L-DOPA or other dopamine precursors are also often administered with compounds that inhibit the action of catechol-O-methyl transferase (COMT inhibitors). Non-limiting examples of COMT inhibitors for use in the methods herein include entacapone, tolcapone, and nitecapone.

The methods of the invention encompass preventing, attenuating, and/or treating any of the movement or motor disorder side effects described herein associated with L-DOPA treatment, or treatment with another dopamine-related drug, L-DOPA treatment or other dopamine-related drug treatment in combination with DDCI (AAADI) treatment, L-DOPA treatment or other dopamine-related drug treatment in combination with COMT inhibitors, and L-DOPA treatment or other dopamine-related drug treatment in combination with DDCI (AAADAI) treatment and COMT inhibitors. In one embodiment, the methods of the invention encompass preventing, attenuating, and/or treating any of the motor disorder side effects described herein, associated with administering the combination of carbidopa, levodopa, and entacapone. In one embodiment, the combination of carbidopa, levodopa, and entacapone is administered as Stalevo.

In an embodiment, the methods of the invention encompass preventing, attenuating, and/or treating PD itself, including the movement disorders associated with PD. In preferred embodiments, the compound having agonist activity at both the 5-HT₁ₐ and 5-HT_{1b} receptors is eltoprazine or a pharmaceutically acceptable salt thereof. In an embodiment, the compound is eltoprazine hydrochloride.

In an embodiment, the invention provides a method of prevention, attenuation, and/or treatment of movement or motor disorder side effects, including but not limited to dyskinesia, that are associated with L-DOPA therapy, such as LID, or other dopamine-related drugs in PD patients comprising administering to a patient in need thereof a therapeutic dose of a compound or a combination of two or more different compounds that acts/act as an agonist or partial agonist at the 5-HT₁ₐ receptor in a dosing regimen involving administering the compound or combination of compounds at certain dosing times and intervals.

In another embodiment, this invention provides a method of prevention, attenuation, and/or treatment of movement or motor disorder side effects, including but not limited to dyskinesia, that are associated with L-DOPA therapy or other dopamine-related drugs in PD patients comprising administering to a patient in need thereof a therapeutic dose of a compound or a combination of two or more different compounds that acts/act as an agonist or partial agonist at the 5-HT_{1b} receptor.

In another embodiment, this invention provides a method of prevention, attenuation, and/or treatment of movement or motor disorder side effects, including but not limited to dyskinesia, that are associated with L-DOPA therapy or other dopamine-related drugs in Parkinson's patients comprising administering to a patient in need thereof a therapeutic dose of a compound or a combination of two or more different compounds that acts/act as an agonist or partial agonist at both the 5-HT₁ₐ and 5-HT_{1b} receptors. When a single compound is used, this compound may act as an agonist or partial agonist at both the 5-HT₁ₐ and 5-HT_{1b} receptors. When two or more different compounds are used, certain compounds may act at solely on one receptor (5-HT₁ₐ or 5-HT_{1b}) and certain compounds may act at both receptors. In one embodiment, each compound acts solely on a particular receptor (5-HT₁ₐ or 5-HT_{1b}) with the combination of compounds used resulting in the activity of both receptors. In another embodiment, at least one compound acts on both receptors. In another embodiment, all compounds act on both receptors.

In one embodiment, this invention provides a method of prevention, attenuation, and/or treatment of movement or motor disorder side effects, including but not limited to dyskinesia or LID, that are associated with L-DOPA therapy or other dopamine-related drugs in Parkinson's patients comprising administering to a patient in need thereof a therapeutic dose of eltoprazine. In a preferred embodiment, the invention provides a method of preventing, attenuating, and/or treating Parkinson's disease, comprising administering to a patient in need thereof a dopamine-related drug and most preferably, L-DOPA, in combination with a therapeutic dose of eltoprazine or a pharmaceutically acceptable acid addition salt thereof.

In some embodiments, the invention provides methods for treating one or more symptoms of Parkinson's disease. Examples of such symptoms include but are not limited to dyskinesia, hyperkinesia, speech changes, loss of facial expression, cognitive dysfunction, mood swings, emotional ability, euphoria, bipolar syndrome, anxiety, aphasia, dysphasia, or disturbances, dementia or confusion, depression, fear, anxiety, memory difficulties, slowed thinking, sexual dysfunction, fatigue, aching, and loss of energy.

For all the conditions described herein, one of ordinary skill in the art will appreciate how to determine the presence or absence of characteristic symptoms and also how to diagnose these conditions. A number of criteria for diagnosing disease are useful for characterizing these conditions such as for example, NINCDS-ADRDA criteria (McKhann et al., 1984), the ICD-IO criteria (World Health Organization, 1992), and/or the DSM-IV criteria (American Psychiatric Association, 1994). Other manuals useful in diagnosing the conditions described herein include for example, but are not limited to Oppenheimer's Diagnostic Neuropathology: A Practice Manual (Esiri and Perl, 2006, Hodder Amold, London.); Harrison's Principles of Internal Medicine (Ed. Kasper et al, 16th Ed. 2005 McGraw Hill, Columbus, Ohio); Goetz: Textbook of Clinical Neurology (Eds. Goetz, Pappert, 2nd Ed. 2003, W.B. Saunders, Philadelphia, Pa.). One of ordinary skill will be aware of other such manuals routinely used in the art to diagnose these conditions.

Eltoprazine (1-(2,3 -dihydro-1, 4-benzodioxanyl-5-yl) piperazine) is particularly preferred for use in the methods of the invention, including pharmaceutically acceptable salts thereof, and preferably HC1. Another preferred compound that may be useful for this invention is batoprazine, (8-(1-piperazine)-2H-1-benzopyran-2-one). This invention also includes the use of prodrugs of the compounds of the formulas provided, specifically derivatives of the compounds of the formulas that are inactive but are converted to an active form in the body following administration.

Eltoprazine and processes for its synthesis are known in the art and is described in U.S. Pat. No. 4,833,142; U.S. Pat. No. 5,424,313; European Patent No. 189,612; and European Patent No. 138,280, each of which is incorporated herein by reference in its entirety. Eltoprazine is commercially available, for example, through Tocris Bioscience (Ellisville, Mo.).

The invention encompasses many possible administration strategies including, but not limited to, administration of the 5-HT_{1a/1b} receptor agonist or partial agonist before or after initiation of L-DOPA or other dopamine-related drug administration, and administration of the 5-HT_{1a/1b} receptor agonist or partial agonist before or after development of motor disorder side effects. Dosage strategies include therapeutic and sub-therapeutic dosages of L-DOPA or other dopamine-related drug. In certain embodiments, this invention encompasses a reduction in the dosage of L-DOPA or other dopamine-related drug after administration of the 5-HT_{1a/1b} receptor agonist or partial agonist. In instances where eltoprazine is used as the 5-HT_{1a/1b} receptor agonist, non-limiting examples of daily dosages include from 3 mg to 30 mg; or 2.5 mg, 5.0 mg, 7.5 mg, and 10 mg as embodiments within the scope of the invention. Particularly preferred dosages of eltoprazine, in humans, are 5 mg/day and 7.5 mg/day, at one or more dosing intervals. Administration schedules may also be altered to achieve a therapeutically effective concentration of compound to treat the disorder or symptoms described herein. In some embodiments, for example, the compound may be administered once per day, twice per day, thrice per day, 4 times per day, 5 times per day, 7 times per day or 10 times per day.

The methods of the invention embrace a number of newly determined, antidyskinetic dosing combinations and regimens involving eltoprazine to reduce LID and/or to optimize the daytime plasma concentration in the individual (human patient) undergoing treatment. In accordance with the present invention, doses of eltoprazine administered to a patient in need thereof in certain dosing schedules or regimens were surprisingly found to be effective in achieving maximal and desired plasma concentrations in the patient with lasting effects. By way of example, daily doses of eltoprazine of 5 mg to 10 mg, preferably 5 mg and 7.5 mg, resulted in a daytime plasma concentration in the range of 10 ng/ml to 30 ng/ml. In an embodiment, the plasma concentration range is 9 ng/ml or 17 ng/ml.

In embodiment, the methods involve administering eltoprazine to a patient in need at a dose of 5 mg or 7.5 mg to effectively reduce dyskinesia, namely, LID. In embodiments, a daytime plasma concentration following eltoprazine administration is in the range of 10 to 30 ng/ml or in the range of 9 to 17 ng/ml. In particular embodiments, at a dose of 5 mg, the plasma concentration of drug (eltoprazine) is approximately 10 ng/ml, while at a dose of 7.5 mg, the plasma concentration of drug (eltoprazine) is approximately 17 ng/ml. The invention encompasses a variety of daily dosing combinations to achieve a desired and effective target daytime plasma concentration range. Oral dosing and administration of eltoprazine are embraced by this dosing combination and regimen.

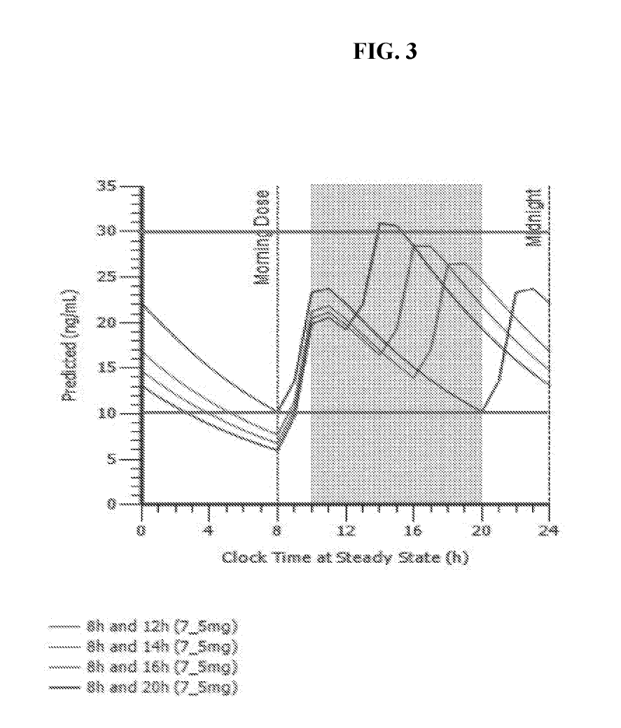

In an embodiment, the methods involve administering eltoprazine to a patient in need twice daily at four or six hour intervals with an eltoprazine dose of 2.5 mg, 5 mg, or 7.5 mg. In a particular embodiment, the dose is 5 mg. In another particular embodiment, the dose is 7.5 mg. In an aspect, dosing at four or six hour intervals may achieve optimal daytime plasma concentration in the individual from 10 AM to 8 PM, for example. See, e.g., FIG. 3. Oral dosing and administration of eltoprazine are embraced by this dosing combination and regimen.

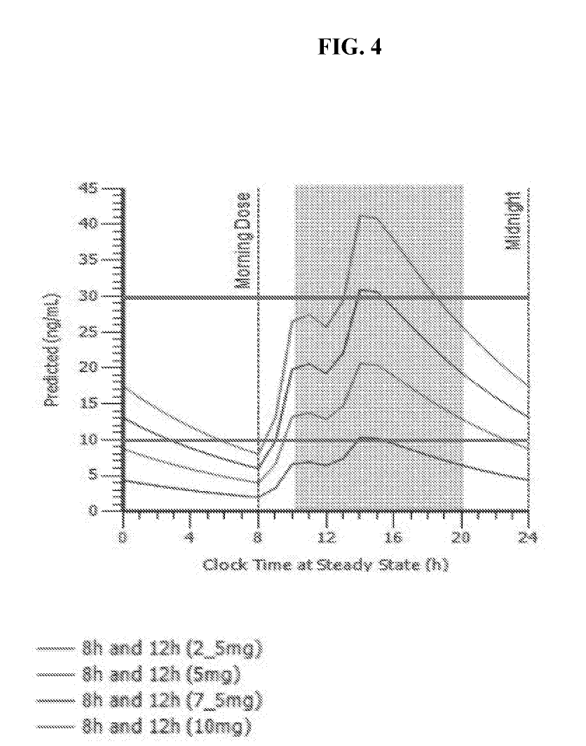

In an embodiment, the methods involve administering eltoprazine to a patient in need twice daily at four hour intervals with an eltoprazine dose of 2.5 mg, 5 mg, 7.5 mg, or 10 mg. In a particular embodiment, the dose is 5 mg. In another particular embodiment, the dose is 7.5 mg. In an aspect, eltoprazine doses of 5 mg and 7.5 mg may optimize the daytime plasma concentration, for example, from 10 AM to 8 PM. See, e.g., FIG. 4. Oral dosing and administration of eltoprazine are embraced by this dosing combination and regimen.

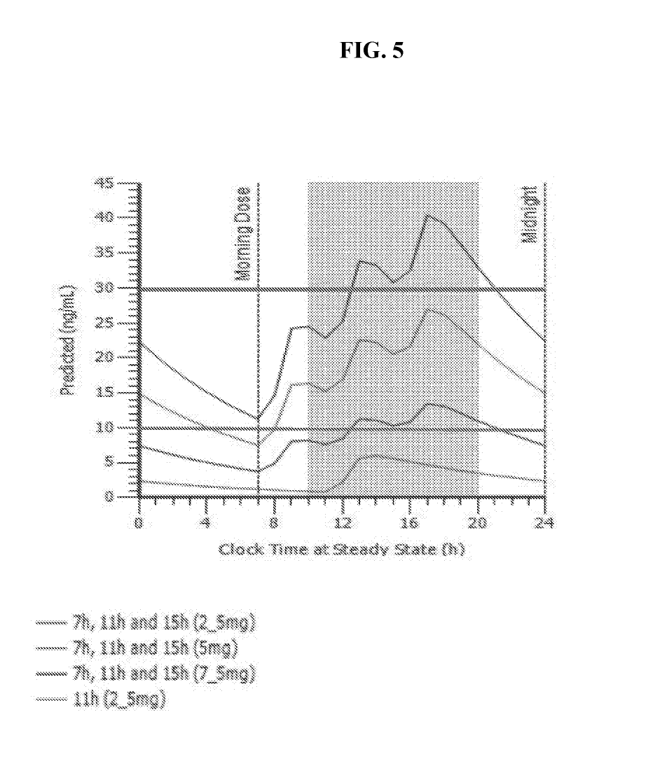

In an embodiment, the methods involve administering eltoprazine to a patient in need three times daily at four hour intervals with an eltoprazine dose of 2.5 mg, 5 mg, or 7.5 mg. In a particular embodiment, the dose is 5 mg. In another particular embodiment, the dose is 7.5 mg. In an aspect, three daily doses of eltoprazine, such as a 5 mg dose and a 7.5 mg dose, may allow maximal plasma concentrations to be achieved, for example, in the late afternoon evening period, extending into the night time, thus providing longer term reduction in the patient's symptoms. See, e.g., FIG. 5. Oral dosing and administration of eltoprazine are embraced by this dosing combination and regimen.

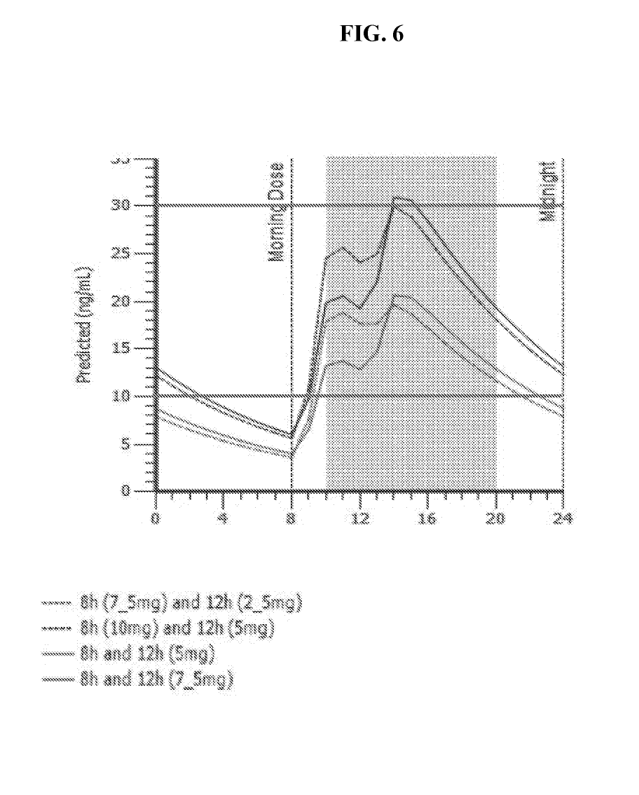

In another embodiment, the methods involve administering a loading dose of eltoprazine to a patient in need, followed by a subsequent dose at a predetermined time thereafter. More particularly, a dose of eltoprazine is administered early in the day, such as a morning loading dose, followed by another dose at a later interval, such as at midday. In embodiments, the subsequent, ‘top up’ dose may be administered one, two, three, four, five, six, seven, eight, nine, ten, eleven, twelve, etc. hours, and intervals there between, after the initial loading dose. By way of example, the subsequent dose may be administered from one to twelve hours after the loading dose; or from two to ten hours after the loading dose; or from two to six hours after the loading dose, etc., A total daily dose of eltoprazine may be 10 mg or 15 mg. Such a regimen involving a loading dose followed by one or more smaller, ‘top up’ doses, may optimize daytime exposure while minimizing the overall dose administered to the individual. See, e.g., FIG. 6. In an embodiment, an individual in need is dosed with a 7.5 mg loading dose of eltoprazine, followed by a 2.5 mg dose of eltoprazine thereafter. In an embodiment, an individual in need is dosed with a 7.5 mg loading dose of eltoprazine, followed by a 5 mg dose of eltoprazine thereafter. In an embodiment, an individual in need is dosed with a 10 mg loading dose of eltoprazine, followed by a 5 mg dose of eltoprazine thereafter. In an embodiment, an individual in need is dosed with a 10 mg loading dose of eltoprazine, followed by a 2.5 mg dose of eltoprazine thereafter. Oral dosing and administration of eltoprazine are embraced by this dosing combination and regimen.

In embodiments related to oral dosing of eltoprazine and the dosing regimens herein described, the oral dosage form is not intended to be limiting and thus may include liquid oral dosing forms and solid oral dosage forms, such as pills, capsules, or tablets. Multilayered tablets, such as bi-layered and tri-layered tablets are encompassed by the invention. Without limitation, controlled release, sustained release, delayed release and extended release solid oral dosage forms are encompassed by the invention.

In accordance with the methods, eltoprazine and/or a related compound(s) is administered in combination with one or more additional compound(s). The additional compound(s) may have actions that are similar to, synergistic to, or different than eltoprazine and/or its related compound(s). Non-limiting examples of additional compounds and drugs that may be administered in combination with eltoprazine may be found, for example and without limitation, in U.S. Pre-Grant Publication No. US 2013/0331399, the contents of which are incorporated by reference herein in their entirety. In an embodiment, eltoprazine and/or a related compound(s) is administered optionally in combination with one or more additional compound(s) for prevention, attenuation, and/or treatment of dyskinesia or other movement or motor disorder or side effect thereof that is associated with L-DOPA therapy or other dopamine-related drugs in Parkinson's patients. In another embodiment, eltoprazine and/or a related compound(s) is administered optionally in combination with one or more additional compound(s) for prevention, attenuation, and/or treatment of PD.

In yet another embodiment, L-DOPA is administered to a PD patient in need thereof, and following this administration, by the methods described herein, eltoprazine and/or a related compound(s) is administered. The eltoprazine and/or a related compound(s) is administered optionally in combination with one or more additional compound(s) for prevention, attenuation, and/or treatment of dyskinesia that is associated with L-DOPA therapy. In yet another embodiment, L-DOPA is administered to a PD patient in need thereof following the administration of eltoprazine and/or a related compound(s) optionally in combination with one or more additional compound(s), for prevention, attenuation, and/or treatment of dyskinesia that is associated with L-DOPA therapy.

In yet another embodiment, L-DOPA is administered to a PD patient in need thereof, and following this administration, by the methods described herein, eltoprazine and/or a related compound(s) is administered after the development of motor side effects, optionally in combination with one or more additional compound(s), for prevention, attenuation, and/or treatment of dyskinesia that is associated with L-DOPA therapy.

In yet another embodiment, L-DOPA is administered to a PD patient in need thereof, and following this administration, by the methods described herein, eltoprazine and/or a related compound(s) is administered prior to the development of motor side effects, optionally in combination with one or more additional compound(s), for prevention, attenuation, and/or treatment of dyskinesia that is associated with L-DOPA therapy.

In one embodiment, the invention is not used for prevention, attenuation, and/or treatment of the associated cognitive impairment in PD. In another embodiment, the invention is not used for prevention, attenuation, and/or treatment of any one or more of the following: Alzheimer's disease, Huntington's disease, Cushing's syndrome, Lewy body disease, multiple sclerosis, stroke, addictive disorders, pervasive development disorder, Fragile X syndrome, anxiety disorders, Prader-Willi Syndrome, schizophrenia, bipolar disorder, depressive disorders, vascular dementia, mild cognitive impairment, dementia, or delirium. In an alternative embodiment, the invention may be used for prevention, attenuation, and/or treatment of one or more of these disorders.

In one embodiment, the methods of the invention encompass preventing, attenuating, and/or treating a movement or motor disorder, or a movement or motor side effect associated with a dopamine-related drug for the treatment of PD, which is not L-DOPA.

In some embodiments, the invention encompasses preventing, attenuating, and/or treating in patients with PD, movement disorders, motor disorders or side effects thereof associated with dopamine-related drugs, in addition to preventing, attenuating, and/or treating PD, by administering to a patient in need thereof a therapeutic dose of a compound having agonist activity at both the 5-HT₁ₐ and 5-HT_{1b} receptors, or two separate compounds having agonist activity, one targeting the 5-HT₁ₐ receptor, and another compound targeting the 5-HT_{1b} receptor, in combination with deep brain stimulation as performed in a surgical procedure, or in combination with magnetic brain stimulation, such as transcranial magnetic stimulation. In one embodiment, the invention encompasses preventing, attenuating, and/or treating in patients with PD, movement disorders, or motor disorder side effects associated with dopamine-related drugs, in addition to preventing, attenuating, and/or treating PD, by administering to a patient in need thereof a therapeutic dose of eltoprazine in combination with deep brain stimulation and/or transcranial magnetic stimulation, and optionally in combination with any other compound described herein. Any order of treatment found to be beneficial is contemplated by the invention. For example, the deep brain stimulation and/or transcranial magnetic stimulation may precede the dopamine-related drug treatment, may follow the dopamine-related drug treatment, may precede the eltoprazine treatment, or may follow the eltoprazine treatment.

The doses of the compounds used in treating the disorders described herein in accordance with this invention will vary in the usual way with the seriousness of the disorder, the weight, and metabolic health of the individual in need of treatment. The methods of the invention provide therapeutically effective doses and treatment schedules for administration to individual patients to arrive at the desired therapeutic or prophylactic effect, including a desired plasma concentration, while minimizing side effects. Particularly beneficial doses of eltoprazine for providing a more sustained effect include a 5 mg dose and a 7.5 mg dose. In a preferred embodiment, the 5 mg and the 7.5 mg doses of eltoprazine are administered to a patient three times daily. In another preferred embodiment, the 5 mg and the 7.5 mg doses of eltoprazine are administered to a patient three times daily at four hour intervals.

Administration schedules may also be altered to achieve other therapeutically effective concentrations of compound to treat the disorders or symptoms described herein.

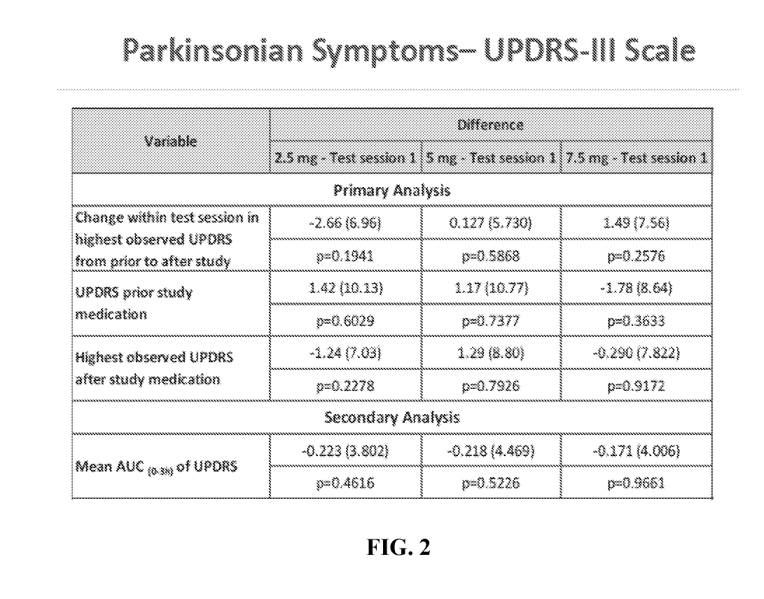

The methods described herein reflect the discovery of particular efficacious doses of eltoprazine and dosing schedules that achieve plasma concentrations in treated individuals for reducing and/or treating levodopa-induced dyskinesia in Parkinson's patients. The particularly efficacious doses and schedules are 5 mg and 7.5 mg of eltoprazine administered two or three times per day at four hour intervals, for example, as shown in FIGS. 4 and 5 herein. Example 1 and FIG. 2 also show that administration of eltoprazine to Parkinson's patients already on levodopa treatment who then receive eltoprazine do not show a reduction in the efficacy of levodopa therapy for treating PD. Further, eltoprazine was well-tolerated, and there were no serious adverse events from administration.

In some embodiments, eltoprazine and/or related compounds may be administered once per day, twice per day, thrice per day, 4 times per day, 5 times per day, 7 times per day or 10 times per day. In preferred embodiments, eltoprazine is administered one, two, or three times per day. Often the dosage is divided equally throughout the day, however in some embodiments to treat certain disorders or symptoms, it may be useful to bias the dosage administration schedule so that most of the daily treatment is administered at the beginning half or portion of the day. In some embodiments, about 50%, 60%, 70% or 80% of the dosage is administered in the first half or portion of the day. In other embodiments, it may be more appropriate to administer most of the dosage in the latter half or portion of the day so that about 50%, 60% , 70% or 80% of the dosage is administered in the latter half or portion of the day.

The 5-HT_{1a/1b} receptor agonist, partial agonist, or agonists and, optionally, at least one additional compound, may be administered before, concurrently, or after administration of L-DOPA or other dopamine-related drug. In one embodiment, the 5-HT_{1a/1b} receptor agonist, partial agonist, or agonists and, optionally, at least one additional compound, is administered before administration of L-DOPA or other dopamine-related drug. In one embodiment, after administration of the 5-HT_{1a/1b} receptor agonist, partial agonist, or agonists and, optionally, at least one additional compound, the dose of L-DOPA or other dopamine-related drug is reduced. The 5-HT_{1a/1b} receptor agonist, partial agonist, or agonists may be administered before, concurrently, or after administration of at least one additional compound.

The 5-HT_{1a/1b} receptor agonist, partial agonist, or agonists and, optionally, at least one additional compound, may be administered before, concurrently, or after onset of symptoms. In one embodiment, the symptoms include a movement or motor disorder, or a movement or motor disorder side effect from the use of L-DOPA or other dopamine-related drugs. In one embodiment, the 5-HT_{1a/1b} receptor agonist and, optionally, at least one additional compound, is/are administered after the development of a movement or motor disorder side effect. In one embodiment, the 5-HT_{1a/1b} receptor agonist, partial agonist, or agonists and, optionally, at least one additional compound, is/are administered before the development of a movement or motor disorder side effect.

Administration of the compounds of this invention may be by any method used for administering therapeutics, such as for example, oral, parenteral, intravenous, intramuscular, subcutaneous, rectal, or topical administration, such as through the use of a transdermal patch. In a particular embodiment, eltoprazine is orally administered to a patient in need.

It will be appreciated by one of ordinary skill in the art that age of the patient with the conditions described herein may respond to treatment at different degrees depending on factors such as dosage or administration or the presence of other factors or co-morbid conditions. Therefore, one of ordinary skill in the art will appreciate that the methods described herein may be directed to a particular age group.

In addition to comprising the therapeutic compounds for use in this invention, especially eltoprazine [1-(2,3-dihydro-1, 4-benzodioxin-5-yl) piperazine] or pharmaceutically acceptable salts (preferably HCl in the case of eltoprazine) or pro-drug thereof, the pharmaceutical compositions for use with this invention may also comprise a pharmaceutically acceptable carrier. Such carriers may comprise additives, such as preservatives, excipients, fillers, wetting agents, binders, disintegrants, buffers may also be present in the compositions of the invention. Suitable additives may be, for example magnesium and calcium carbonates, carboxymethylcellulose, starches, sugars, gums, magnesium or calcium stearate, coloring or flavoring agents, and the like. There exists a wide variety of pharmaceutically acceptable additives for pharmaceutical dosage forms, and selection of appropriate additives is a routine matter for those skilled in art of pharmaceutical formulation.

The compositions for use in the methods may be in the form of tablets, capsules, powders, granules, lozenges, suppositories, reconstitutable powders, or liquid preparations such as oral or sterile parenteral solutions or suspensions.

In order to obtain consistency of administration it is preferred that a composition of the invention is in the form of a unit dose. Unit dose forms for oral administration may be tablets, capsules, and the like, and may contain conventional excipients such as binding agents, for example syrup, acacia, gelatin, sorbitol, tragacanth, or polyvinylpyrrolidone; and carriers or fillers, for example, lactose, sugar, maize-starch, calcium phosphate, sorbitol or glycine. Additives may include disintegrants, for example starch, polyvinylpyrrolidone, sodium starch glycolate or microcrystalline cellulose; preservatives, and pharmaceutically acceptable wetting agents such as sodium lauryl sulfate.

In addition to unit dose forms, multi-dosage forms are also contemplated to be within the scope of the invention. Modified or controlled release dosage forms are contemplated for use in the invention, including, but not limited to, controlled release dosage forms, sustained release dosage forms, extended release dosage forms, delayed release dosage forms, and pulsatile release dosage forms.

In an embodiment, the 5-HT_{1a/1b} receptor agonist, such as eltoprazine or a pharmaceutically acceptable salt thereof, is administered in an oral solid dosage form. The oral solid dosage form may be a pill, capsule, caplet, or tablet. In an embodiment, eltoprazine is administered in the form of a multi-layered tablet, such as a bi-layered or a tri-layered tablet. The methods encompass the administration of eltoprazine as the active ingredient in a bi- or tri-layered oral solid dosage form as a single daily dose, as described herein.

Suitable polymers for use in the controlled release formulations of the present invention include, but are not limited to uncrosslinked, linear polymers including cellulosic polymers, preferably hydroxyethyl cellulose, sodium carboxymethyl cellulose, hydroxypropylmethyl cellulose and hydroxypropyl cellulose, microcrystalline cellulose, methyl cellulose, and ethyl cellulose, and combinations thereof; covalently crosslinked insoluble polymers such as high molecular weight crosslinked homopolymers and copolymers of (meth) acrylic acid including carbopol resins, or mixtures of these uncrosslinked and covalently crosslinked polymers. Additionally suitable polymers include acrylic acid, methacrylic acid, methyl acrylate, ammonio methylacrylate, ethyl acrylate, methyl methacrylate and/or ethyl methacrylate, vinyl polymers and copolymers such as polyvinyl pyrrolidone, polyvinyl acetate, polyvinylacetate phthalate, vinylacetate crotonic acid copolymer, and ethylene-vinyl acetate copolymers, to name a few. Various combinations of two or more of the above polymers are also contemplated for use in the dosage forms of the invention.

Delayed release compositions may be prepared, for example, by employing slow release coatings, micro encapsulation, and/or slowly dissolving polymers.

The solid oral compositions may be prepared by conventional methods of blending, filling, tabletting or the like. Repeated blending operations may be used to distribute the active agent throughout those compositions employing large quantities of fillers. Such operations are conventional in the art. The tablets may be coated according to methods well known in normal pharmaceutical practice, for example, with an enteric coating.

Multilayered tablets may be produced by processes and procedures known and available in the art. See, e.g., EP 1681051 and EP 2517696 A1, as relates to the following. In some cases, the tablet comprises multiple layers having different time release profiles. The tablet may have two, three, four or five layers, in which, for example, the first layer is formulated for immediate release and the second layer is formulated for delayed release. In embodiments having a third layer, the third layer may be formulated to have a release that is later than the second layer. In some embodiments having a third layer, the third layer is located in the middle of the tablet. In other embodiments having multiple layers, the layers having the longest delay are formulated to be nearest the center of the multi-layer tablet, either in horizontal layers, or in the form of a core coated by subsequent layers.

In certain embodiments comprising a layer formulated for immediate release, such a layer is formulated with one or more rate-controlling polymers and pharmaceutically acceptable excipients generally known in the art for immediate release. In certain embodiments comprising a layer formulated for delayed release, such a layer is formulated with one or more rate-controlling polymers and pharmaceutically acceptable excipients generally known in the art for immediate release. In certain embodiments of a delayed release layer, the rate-controlling polymer is a polyethylene oxide polymer. The molecular weight of the polyethylene oxide molecule of the polyethylene oxide polymer used may be 7,000,000 g/mole and Brookfield viscosity of its 1% m/v aqueous solution at 25° C. is 7500-10000 cps (Colorcon-Sentry Polyox WSR 303-Leo-NF-Dow).

In certain embodiments, the tablet developed is composed of two layers, with one of the layers providing immediate release, and the other layer providing extended release. By way of example, 65-85% of the active ingredient (API) content of the tablet may be present at the extended release providing layer and 15-35% may be present at the immediate release providing layer. The two separate layers may be compressed as two layer tablet. In another embodiment, the layer providing extended release forms the tablet core and the layer providing immediate release is coated on the tablet core. In another exemplary embodiment of a two layer tablet, the immediate release layer provides extended release between 20% to 40% in the first hour in order to provide the adequate effective blood level and two-phase pellets to achieve Cₘₐₓ value. Thereafter, in in order to keep the plasma concentration of the active ingredient effective in the long run, it provides 8-hour and longer term release of the remaining active ingredient amount. In a dissolution test, 20% to 40% of the active ingredient is released in the first hour at 0.1 N HCl medium. The other part of the composition provides 5% to 12% release per hour in pH 6.8 phosphate buffer.

Excipients known in the art may be used to formulate the extended release providing layer, including, but not limited to, calcium phosphate dibasic as buffer agent, talc and magnesium stearate as lubricant and colloidal silicon dioxide (Aerosil 200) as glidant. Excipients known in the art may be used to formulate the immediate release providing layer, including, but not limited to, microcrystalline cellulose as diluent and binder, croscarmellose sodium as disintegrant, magnesium stearate as lubricant and colloidal silicon as glidant.

Rate controlling polymers employed in multi-layered tablet forms containing one or more active ingredients may be one or more of hydrophilic polymers, hydrophobic polymers or a combination thereof. The one or more rate-controlling polymers may comprise about 5% to about 60% w/w of the tablet. Hydrophilic polymers in multi-layer tablet forms may be one or more of cellulose derivatives comprising hydroxypropylcellulose, hydroxypropyl methylcellulose, hydroxyethylcellulose, hydroxymethylcellulose, carboxymethylcellulose, methylcellulose, sodium carboxymethylcellulose or combinations thereof; polyvinylpyrrolidone, vinyl acetate/vinyl-pyrrolidone copolymer, microcrystalline cellulose, polysaccharides, polyalkylene glycols, starch and derivatives thereof. The hydrophobic polymers in multi-layer tablet forms may be one or more of ethyl cellulose, cellulose acetate, cellulose acetate butyrate, hydroxypropyl methylcellulose phthalate, poly (alkyl) methacrylate, and copolymers of acrylic or methacrylic acid esters, waxes, shellac and hydrogenated vegetable oils. The layers may be prepared by one or more of dry granulation, wet granulation or direct compression. The layer containing the active ingredient may further include an alkalizing agent. The tablet may further include one or both of an outer protective coating layer or a separating layer between first and second layers. The tablet may exhibit a Tₘₐₓ of one active which occurs at a time 4 hours to about 10 hours and exhibits a Tₘₐₓ of another active which occurs at a time 5 hours to about 18 hours after administration of the tablet to a human patient. See, e.g., WO 2006092711 A2.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol syrup, methyl cellulose, gelatin, hydroxyethylcellulose, carboxymethylcellulose, aluminum stearate gel, and hydrogenated edible fats; emulsifying agents, for example lecithin, sorbitan monooleate, or acacia; non-aqueous vehicles (which may include edible oils), for example almond oil or fractionated coconut oil, oily esters such as esters of glycerine, propylene glycol, or ethyl alcohol; preservatives, for example methyl or propyl p-hydroxybenzoate or sorbic acid; and if desired conventional flavoring or coloring agents.

For parenteral administration, fluid unit dosage forms are prepared utilizing the compound and a sterile vehicle, and, depending on the concentration used, can be either suspended or dissolved in the vehicle. In preparing solutions, the compound can be dissolved in water or saline for injection and filter sterilized before filling into a suitable vial or ampoule and sealing. Advantageously, additives such as a local anesthetic, preservative and buffering agent can be dissolved in the vehicle. Suitable buffering agents are, for example, phosphate and citrate salts. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilized by conventional means, for example by exposure to radiation or ethylene oxide, before being suspended in the sterile vehicle. Advantageously, a surfactant or wetting agent is included in the composition to facilitate uniform distribution of the compound.

The 5-HT_{1a/1b} receptor agonist, partial agonist, or agonists and, optionally, at least one additional compound may be provided in a kit. In one embodiment, a kit may comprise at least one 5-HT_{1a/1b} receptor agonist, partial agonist, or agonists, and at least one additional compound. In one embodiment, a kit may comprise at least one 5-HT_{1a/1b} receptor agonist, partial agonist, or agonists, L-DOPA or other dopamine-related drug, and optionally at least one additional compound, such as set forth, for example, in U.S. Pre-Grant Publication No. US 2013/0331399, the contents of which are incorporated by reference herein in their entirety. In an embodiment, the kit may also include instructions for administration of the compounds. In one embodiment, the kit is intended for use by a subject having PD. In another embodiment, a kit may comprise at least one 5-HT_{1a/1b} receptor agonist, partial agonist, or agonists, at least one dopamine precursor, at least one DDCI, and instructions for administering the compounds. In another embodiment, a kit may comprise at least one 5-HT_{1a/1b} receptor agonist, partial agonist, or agonists, at least one dopamine precursor, at least one DDCI, at least one COMT inhibitor, and instructions for administering the compounds. In an embodiment, the at least one 5-HT_{1a/1b} receptor agonist is eltoprazine.

All patents and patent publications referred to herein are hereby incorporated by reference.

Certain modifications and improvements will occur to those skilled in the art upon a reading of the foregoing description. It should be understood that all such modifications and improvements have been omitted herein for the sake of conciseness and readability but are properly within the scope of the following claims. It is understood that the following examples and embodiments described herein are for illustrative purposes only and that various modifications or changes in light thereof will be suggestive to persons skilled in the art and are to be included within the spirit and purview of this application and the scope of the appended claims.

### Example 1

### Treatment of motor disorder side effects associated with L-DOPA therapy using eltoprazine in human patients.

This example describes a multicenter, randomized, double-blind, placebo-controlled, dose finding study of oral eltoprazine in Parkinson's patients with L-DOPA induced dyskinesias, in a levodopa challenge-dose setting in Parkinson's Disease. A total of 22 patients participated in the study, out of which 18 patients fulfilled the protocol in terms of eligibility, interventions, and outcome assessment.

### Inclusion Criteria:

Subjects had to meet all of the following criteria to be eligible for the study:

1. Each patient had Parkinson's disease, defined according to the UK Brain Bank Criteria (see Hughes et al., J Neurol Neurosurg Psychiatry, 1992. 55(3): p. 181-4; Lees et al., Lancet, 2009. 373: p. 2055-66). Bradykinesia symptoms were combined with one of resting tremor, rigidity or postural imbalance.
2. Each patient had been treated for at least 3 years with L-DOPA prior to this study.
3. Each patient had significant dyskinesias after L-DOPA dosages according to clinical experience.
4. Each patient had significant dyskinesias after the administration of a single challenge dose of L-DOPA using two dyskinesia rating scales. The screening test was performed with a challenge dose of 150% of the normal regular dose of L-DOPA. If no dyskinesias was present during the first challenge, the challenge was repeated on an alternate day (and) significant dyskinesias in a patient self-administered diary with 3 levels with 3 grades (“off,” “on without dyskinesias,” “on with hyperkinesias”) after the challenge dose has been given. Patients were included if one of the two challenge tests were positive and diary was positive for dyskinesias. In addition to the observed types of dyskinesias (dystonia, hyperkinesias), a temporal patterns were described as “peak-of dose,” “end-of-dose dyskinesias” and “bi-phasic dyskinesias.” Quantification was made by “peak-of-dose” ratings and area-under-the-curve (AuC) of scores.
5. Each patient was over 18 years of age.
6. This inclusion criterion applied to females of child-bearing potential (not surgically sterilized and between menarche and 1 year postmenopausal) only. Each female patient tested negative for pregnancy at the time of enrollment based on a serum pregnancy test and agreed to use a reliable method of birth control during the study.
7. Each patient signed informed consent.
8. Each patient had to able to communicate effectively with the investigator and study coordinator.

### Exclusion Criteria:

The presence of any of the following conditions excluded a subject from the study:

1. Exclusion criteria for Parkinson's disease according to the UK Brain Bank Criteria for Parkinson's disease.
2. Fulfillment of any other atypical parkinsonism diagnosis according to published criteria for multiple system atrophy (see Gilman, S., et al., Neurology, 2008. 71(9): p. 670-6), progressive supranuclear paresis (see Litvan, I., et al., Neurology, 1996. 47(1): p. 1-9), dementia with Lewy body (see McKeith, I. G., et al., Neurology, 2005. 65(12): p. 1863-72), corticobasal gangliotic disease (see Litvan, I., et al., J Neuropathol Exp Neurol, 1996. 55(1): p. 97-105), and dementia with Parkinson's disease (see Emre, M., et al., Mov Disord, 2007. 22(12): p. 1689-707; quiz 1837).
3. Any suspected secondary parkinsonism; drug induced parkinsonism; toxin-induced parkinsonism; trauma-induced parkinsonism; normal pressure hydrocephalus and vascular parkinsonism.
4. Ongoing treatment with any selective serotonin re-uptake inhibitors (SSRI) or any combined serotonin-norepinephrine re-uptake inhibitors (SNRT) 4 weeks prior to the study.
5. Ongoing treatment with anti-parkinsonism medications (Cabergoline; Duodopa infusion; ApoGo infusion; Amantadine; Memantine if used against dyskinesias), and other medication with the potential for drug-interactions.
6. Significant depression defined as >18 in the Montgomery Asberg Depression Rating Scale combined with a clinical evaluation as to any clinical relevant depression.
7. Pregnant or breast-feeding.
8. Reduced kidney function; defined as a creatinine level>120 μmol/L.
9. Reduced liver function, defined as aspartate aminotransferase, ASAT>1.0 μkat/L or alanine aminotransferase, ALAT>1.0 μkat/L or glutamyl transpeptidase, GT>1.6 μkat/L, or total bilirubin>30 μmol/L, or alkaline phosphatase, ALP>6.0 μkat/L.
10. History of any other medical condition thought to interfere with the study or study medication (i.e., recent myocardial infarction, uncontrolled diabetes, uncontrolled hypertension (systolic blood pressure>180 mmHg), ongoing severe infection).
11. Receipt of an investigational drug within 30 days or 5 half-lives of the drug, whichever is longer, prior to entering this study.
12. Any known allergy to eltoprazine or the constituents of the study medication and the placebo capsules.
13. Any indication that patients are unsuitable in any other way to participate in this study, in the opinion of the investigator.

This study also assessed the safety and tolerability of eltoprazine in adults with Parkinson's Disease using the following measures: a) population mean values for the change in dyskinesia ratings between the placebo and screening baseline values and any of the eltoprazine dosages used, calculated as the peak-effect on CDRS of any study medication; b) population mean values for the change in dyskinesia ratings between the placebo and screening baseline values and any of the eltoprazine dosages used, calculated as the AuC on Rush DRS of any study medication; c) any changes in the UPDRS-III total score; d) any change in the diary data set, between the baseline and placebo and any of the three study medication tests; e) any deterioration of the HADS scores after study medication compared with placebo; f) any development of depression over the course of the study period, determined by the Montgomery Asberg Depression Rating Scale (MADRS) and clinical judgment; g) comparison between the effects on Rush Dyskinesia Rating Scale (DRS) and Clinical Dyskinesia Rating Scale (CDRS) Area under the Curve (AuC) and peak of dose effects for the three study medication dosages.

The study consisted of 7 visits, described below: a screening visit, five treatment visits, and one end-of-study visit.

### Screening Period (Visit 1)

During the first visit, patients underwent screening for inclusion/exclusion criteria and safety assessments. Symptoms of parkinsonism, depression, and anxiety were assessed, and screening for significant L-DOPA dyskinesias were conducted using a challenge dose (150%) of L-DOPA. Subjects who were taking a prohibited medication were required to complete a washout period of appropriate length, as determined by the investigator. All patients received challenge doses (150%—up to a maximum of 250 mg) of L-DOPA at screening and at each treatment visit. L-DOPA was administered as Sinemet (L-DOPA combined with carbidopa in a fixed ratio of 4:1, unless a patient has a known allergy or intolerance to this drug). If a patient is intolerant to Sinemet, an equivalent dose of Madopar Quick could be used. There was an observation period of 3 hours (6×30 min, or 180 minutes) after dosing.

### Double-Blind Treatment Period (Visits 2-6)

During each of five visits, and after a two-hour fast, each patient received a challenge dosage (150%—up to a maximum of 250 mg) of levodopa. Additionally, during each of the five treatment visits, patients were also treated with single dose treatments of oral capsules of three active study medication dosages (2.5 mg, 5 mg, or 7.5 mg of eltoprazine) or two placebo doses. The patients were periodically recorded for 180 minutes after treatment, and the videos were evaluated in a blinded manner.

Visit 2 occurred within 30 days of visit 1, and visits 3-6 followed one week apart from each other.

### Final Visit or Early Termination Visit (Visit 7)

During the final visit or early termination visit, symptoms of parkinsonism, depression, anxiety, and L-DOPA dyskinesias were assessed, to capture any eltoprazine-related treatment effects on the degree of parkinsonism and any change in mood-related symptoms. Safety assessments were also conducted. A patient diary completed prior to and after the study period was used to evaluate any changes in perceived dyskinesias by the patients. A two-three day diary with 3 symptom lines—“off,” “on (normal),” “on with dyskinesias” were filled out by each patient between the screening and enrollment visits and in between visits 2 through 6 to evaluate any changes in perceived dyskinesia by the patients.

The final visit, 7, was scheduled 4 days after visit 6. All study visits occurred within a ±5-day window of the time points noted above.

After screening, each patient participated in the study for approximately 6-10 weeks. The duration of the study was about 30 weeks. Safety evaluations were based on reports of adverse effects, concomitant therapy, clinical laboratory results, medical history, physical examination, and vital signs. Patient videos were used to assess efficacy. Rating scales include UPDRS, CDRS, and Rush DRS. In addition, patient diaries were used for self-assessment of dyskinesia.

### Rating Scales

The term “Unified Parkinson's Disease Rating Scale-III” and “UPDRS-III” refer to a standardized tool used to measure Parkinson's Disease severity, as described by Fahn et al., in Recent Developments in Parkinson's Disease, Fahn et al. (eds.) Plurham Park, N.J.: Macmillian Healthcare Information, 2:153-163,1987.

The term “Clinical Dyskinesia Rating Scale” and “CDRS” refer to a modified abnormal involuntary movement scale (AIMS) allowing for independent rating of limbs, trunk, head/neck and face rated during the UPDRS movements. This scale can simultaneously rate dystonia and dyskinesias, as described by Goetz et al., in Movement Disorders, Vol. 9, No. 4, 1994, 390-394.

The term “Rush Dyskinesia Rating Scale” and “Rush DRS” refer to an observer-based rating scale based on fixed movements. The numerical parts of the Rush DRS are recorded separately from the descriptive parts, as described by Goetz et al., in Movement Disorders, Vol. 9, No. 4, 1994, 390-394.

The term “Hospital Anxiety & Depression Scale” and “HADS” refer to rating scales commonly used by doctors to determine the levels of anxiety and depression that a patient is experiencing, as described by Zigmond et al., in Acta Psychiatrica Scandinavica 67 (6): 361-370.

The term “Montgomery-Åsberg Depression Rating Scale” and “MADRS” refer to an observer based ten-item diagnostic questionnaire used by psychiatrists to measure the severity of depressive episodes in patients with mood disorders, as described by Montgomery et al., in British Journal of Psychiatry 134 (4): 382-89.

Patients presenting clinically with motor disorder side effects associated with L-DOPA therapy, including dyskinesia, were evaluated using the United Parkinson's Disease Rating Scale III (Recent Developments in Parkinson's Disease, vol. 2, Fahn et al. editors, Macmillan Publishing Co. Inc, 1987), an art-recognized dyskinesia severity scale (Marconi et al., Mov Disord, 9:2-12, (1994)). Briefly, the dyskinesia severity scale rates abnormal movements from 0 (none) to 4 (severe with markedly impaired function) in six different parts of the body (face, neck, and trunk, and four limbs).

### Efficacy Assessments

### Filming

Filming occurred 30 minutes prior to, and every 30 minutes up to 180 minutes after a challenge test of L-DOPA and study medication. Approximately 5 minutes of video filming were performed each time. The “UPRDS movements” in the UPDRS scale was performed (arms in different positions, repeated pronations-supinations of the wrists, finger tapping of opposing fingers, opening and closing of the fists, foot stamping, raising from the chair, walking with a turn and a balance test). Each patient also performed tasks for the “Dyskinesia Rating Scales.” The same sequences were repeated prior to any challenge dose, and after every 30 minutes up to 180 minutes after the intake of the medications, for a total of 7 sequences. Each sequence was rated by two independent blinded raters using UPDRS, CDRS and Rush DRS. Videotaped records of each sequence were assessed by separate individual qualified raters at both of the two clinical centers.

### Patient Diary

During screening, patients were asked to perform a self-administered rating of their dyskinesias by using a diary with 3 levels with 3 grades (“off,” “on without hyperkinesias,” and “on with hyperkinesias”). On the days following screening, the patients self-rated their symptoms over three days. A two day diary with 3-symptom lines (“off,” “on,” “on with dyskinesias”) was filled out by patients between the screening and enrollment visits and in between the Visits 2 through 6 (one day before dosing and one day after dosing).

### Efficacy Analyses

Analyses were done on both the intention-to-treat (ITT) and per protocol (PP) population, if applicable. The change between test sessions of the highest observed Unified Parkinson's Disease Rating Scale-III (UPDRS) within each session will be calculated between the randomized placebo test session and each active test session and will be analyzed with Wilcoxon Signed Rank test on the ITT population.

The change in Mean AUC_{0-3 hours} of CDRS ratings will be analyzed between the randomized Placebo Test session and each active Test session with Wilcoxon Signed Rank test on the ITT population. Changes to all secondary variables between the randomized placebo test session and each active test session were analyzed using Wilcoxon Signed Rank test on both ITT- and PP-population.

### Results

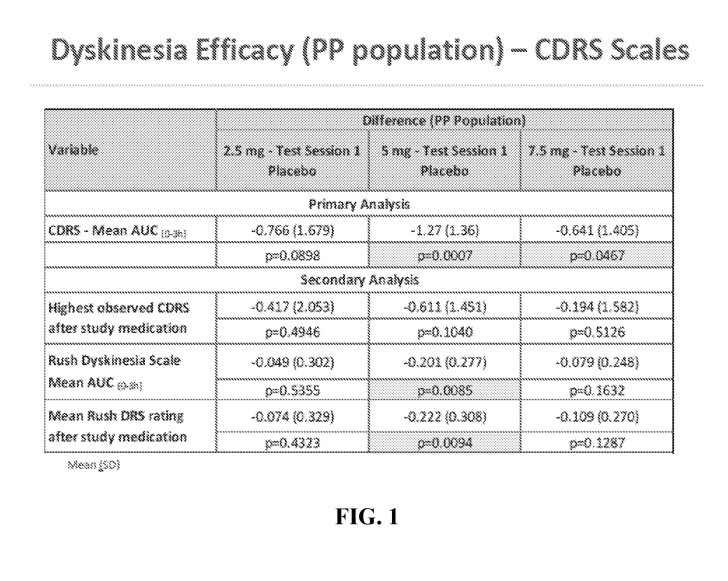

Statistical analyses demonstrated that eltoprazine administered to Parkinson's disease patients with levodopa-induced dyskinesia (LID), statistically significantly reduced LID at both the 5 mg dose (p=0.0007) and the 7.5 mg dose (p=0.0467), as compared to placebo, when measured by the CDRS scale (See, FIG. 1). In addition, eltoprazine administration at these doses did not affect L-DOPA efficacy, as measured by the UPDRS score (See, FIG. 2); both the 5 mg and 7.5 mg doses were statistically significant as compared to placebo). At the 5 mg dose, eltoprazine reduced LID in PD patients statistically significantly—as measured by the Rush Scale AUC.

In addition to its beneficial antidyskinetic effects, eltoprazine was well tolerated in this study, as there were no serious adverse events and normal motor responses to L-DOPA were not altered.

All references, including patent applications and publications cited herein, are incorporated by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A method of treating a movement or motor disorder side effect associated with administration of levodopa to a patient having Parkinson's disease, the method comprising:
administering to said patient in need of treatment a dose of eltoprazine or a pharmaceutically acceptable acid addition salt thereof to reduce said movement or motor disorder side effect, wherein said eltoprazine is administered at a dose of 5 mg or 7.5 mg two or three times per day.

2. The method according to claim 1, wherein said movement or motor disorder side effect is dyskinesia or levodopa-induced dyskinesia.

3. The method according to claim 1 or claim 2, wherein said eltoprazine is administered at a dose of 5 mg two times daily.

4. The method according to claim 1 or claim 2, wherein said eltoprazine is administered at a dose of 5 mg three times daily.

5. The method according to claim 1 or claim 2, wherein said eltoprazine is administered at a dose of 7.5 mg two times daily.

6. The method according to claim 1 or claim 2, wherein said eltoprazine is administered at a dose of 7.5 mg three times daily.

7. The method according to any one of claims 1 to 6, wherein said eltoprazine is administered at four, six, eight and twelve hour intervals.

8. The method according to any one of claims 1 to 6, wherein said eltoprazine is administered at four or six hour intervals.

9. The method according to claim 8, wherein said eltoprazine is administered at four hour intervals.

10. The method according to claim 1 or claim 2, wherein said eltoprazine is administered at a loading dose, followed by administration of a subsequent dose at a predetermined time thereafter.

11. The method according to claim 10, wherein said eltoprazine is administered at a loading dose of 7.5 mg, followed by administration of a subsequent dose of 2.5 mg at from two to twelve hours thereafter.

12. The method according to claim 10, wherein said eltoprazine is administered at a loading dose of 7.5 mg, followed by administration of a subsequent dose of 2.5 mg at from two to six hours thereafter.

13. The method according to any one of claims 1 to 12, wherein said patient is human.

14. The method according to any one of claims 1 to 13, wherein said eltoprazine administration does not reduce efficacy of said levodopa administration.

15. A method of treating Parkinson's disease in a human in need thereof, the method comprising
administering to said human a dose of levodopa and a dose of eltoprazine or a pharmaceutically acceptable acid addition salt thereof, wherein said eltoprazine is administered at a dose of 5 mg or 7.5 mg two or three times per day.

16. The method according to claim 15, wherein said eltoprazine and said levodopa are concurrently active in said human.

17. The method according to claim 15 or claim 16, wherein said eltoprazine is administered to the human before said human develops dyskinesia associated with said levodopa administration.

18. The method according to claim 15 or claim 16, wherein said eltoprazine is administered to said human after said human develops dyskinesia associated with said levodopa administration.

19. The method according to claim 15 or claim 16, wherein said eltoprazine is administered to the human before the administration of said levodopa.

20. The method according to any one of claims 15 to 19, wherein said eltoprazine is administered at a dose of 5 mg two times daily.

21. The method according to any one of claims 15 to 19, wherein said eltoprazine is administered at a dose of 5 mg three times daily.

22. The method according to any one of claims 15 to 19, wherein said eltoprazine is administered at a dose of 7.5 mg two times daily.

23. The method according to any one of claims 15 to 19, wherein said eltoprazine is administered at a dose of 7.5 mg three times daily.

24. The method according to any one of claims 15 to 23, wherein said eltoprazine is administered at four, six, eight and twelve hour intervals.

25. The method according to any one of claims 15 to 23, wherein said eltoprazine is administered at four or six hour intervals.

26. The method according to claim 25, wherein said eltoprazine is administered at four hour intervals.

27. The method according to any one of claims 1 to 26, wherein said eltoprazine is orally administered.

28. The method according to claim 27, wherein said eltoprazine is administered in a controlled release, sustained release, extended release, delayed release, or pulsatile release solid oral dosage form.

29. The method according to claim 28, wherein the solid oral dosage form is a multi-layered tablet dosage form.

30. The method according to claim 29, wherein the solid oral dosage form is a bi-layered or tri-layered tablet dosage form.

31. The method according to claim 30, wherein the bi-layered or tri-layered tablet dosage form is a controlled release, sustained release, extended release, or delayed release oral dosage form.

32. The method according to any one of claims 1 to 14, wherein the movement or motor disorder side effect associated with administration of levodopa is L-DOPA induced dyskinesia (LID).

33. The method according to any one of claims 15 to 26, wherein L-DOPA induced dyskinesia (LID) is treated in the human.

34. The method according to any one of claims 1 to 33, wherein, following eltoprazine administration, plasma concentration of the subject is in the range of 10 ng/ml to 30 ng/ml.

35. The method according to any one of claims 1 to 33, wherein, following eltoprazine administration, plasma concentration of the subject is in the range of 9 ng/ml to 17 ng/ml.
